(19) United States
(12) Patent Application Publication
(10) Pub. No.: US 2008/0019935 A1
(43) Pub. Date: Jan. 24, 2008

(54) Conditioning composition comprising silicone agent for ease-to-rinse feel and/or clean feel

(75) Inventor: Golam Faruque Khan, Darien, CT (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/879,557

(22) Filed: Jul. 18, 2007

Related U.S. Application Data
(60) Provisional application No. 60/831,797, filed on Jul. 19, 2006.

(51) Int. Cl. A61K 8/72; A61Q 5/12

(52) U.S. Cl. 424/70.12

(57) ABSTRACT

Disclosed is a conditioning composition comprising by weight: (a) from about 0.1% to about 10% of a cationic surfactant; (b) from about 1% to about 15% of a high melting point fatty compound; (c) from about 0.01% to about 10% of a silicone agent comprising a silicone backbone, a hydrophilic portion, and a hydrophobic portion; and (d) an aqueous carrier. The composition of the present invention can provide ease-to-rinse feel and/or clean feel, while maintaining conditioning benefits such as slippery feel during the application to wet hair and moisturized feel on dry hair.

This application claims the benefit of U.S. Provisional Application No. 60/831,797, filed on Jul. 19, 2006.

### FIELD OF THE INVENTION

The present invention relates to a conditioning composition comprising: (a) from about 0.1% to about 10% of a cationic surfactant; (b) from about 1% to about 15% of a high melting point fatty compound; (c) from about 0.01% to about 10% of a silicone agent comprising a silicone backbone, a hydrophilic portion, and a hydrophobic portion; and (d) an aqueous carrier. The composition of the present invention can provide ease-to-rinse feel and/or clean feel, while maintaining conditioning benefits such as slippery feel during the application to wet hair and moisturized feel on dry hair.

### BACKGROUND OF THE INVENTION

A variety of approaches have been developed to condition the hair. A common method of providing conditioning benefit is through the use of conditioning agents such as cationic surfactants and polymers, high melting point fatty compounds, low melting point oils, silicone compounds, and mixtures thereof. Most of these conditioning agents are known to provide various conditioning benefits. For example, some cationic surfactants, when used together with some high melting point fatty compounds, are believed to provide a gel matrix which is suitable for providing a variety of conditioning benefits such as slippery feel during the application to wet hair and moisturized feel on dry hair.

There is a need for hair conditioning compositions which provide improved ease-to-rinse feel and/or clean feel, while maintaining conditioning benefits of gel matrix. Hair conditioning compositions containing a gel matrix provide slippery feel during rinsing the hair, even after rinsing the hair. Ease-to-rinse feel and/or clean feel are, for example, fast reduced slippery feel and/or increased clean feel after starting to rinse the hair. Such reduced slippery feel, and/or increased clean feel can be a signal for consumers to stop rinsing the hair conditioning compositions. Consumers who prefer such signals may keep rinsing the hair treated with hair conditioning composition by a large amount of water until they feel the signals. However, such rinsing activity provides less deposition of conditioning agents on the hair, thus less conditioning benefits to the dry hair.

One method of obtaining ease-to-rinse feel and/or clean feel is the reduction of gel matrix and/or conditioning agents. However, such reduced gel matrix and/or conditioning agents provides reduced conditioning benefits, especially reduced gel matrix provides reduced slippery feel during the application.

Thus, there remains a need for hair conditioning compositions which provides such signals faster after starting to rinse the hair, while maintaining wet conditioning benefits of the gel matrix such as slippery feel during the application, and while remaining sufficient amount of deposition of conditioning agents on the hair.

Based on the foregoing, there remains a need for conditioning compositions which provide improved ease-to-rinse feel and/or clean feel, while maintaining improved conditioning benefits such as slippery feel during the application to wet hair and moisturized feel on dry hair.

None of the existing art provides all of the advantages and benefits of the present invention.

### SUMMARY OF THE INVENTION

The present invention is directed to a conditioning composition comprising by weight:

(a) from about 0.1% to about 10% of a cationic surfactant;
(b) from about 1% to about 15% of a high melting point fatty compound;
(c) from about 0.01% to about 10% of a silicone agent comprising a silicone backbone, a hydrophilic portion, and a hydrophobic portion; and
(d) an aqueous carrier.

The conditioning compositions of the present invention can provide improved ease-to-rinse feel and/or clean feel, while maintaining conditioning benefits such as slippery feel during the application to wet hair and moisturized feel on dry hair. The conditioning compositions of the present invention can further provide wet softness and increased rheology in addition to the above improved ease-to-rinse feel and/or clean feel, while maintaining conditioning benefits such as slippery feel during the application to wet hair and moisturized feel on dry hair.

These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

### Composition

The present invention is directed to a conditioning composition comprising by weight:

(a) from about 0.1% to about 10% of a cationic surfactant;
(b) from about 1% to about 15% of a high melting point fatty compound;
(c) from about 0.01% to about 10% of a silicone agent comprising a silicone backbone, a hydrophilic portion, and a hydrophobic portion; and
(d) an aqueous carrier.

The conditioning compositions of the present invention can provide improved ease-to-rinse feel and/or clean feel, while maintaining conditioning benefits such as slippery feel during the application to wet hair and moisturized feel on dry hair. The conditioning compositions of the present invention can further provide wet softness and increased rheology in addition to the above improved ease-to-rinse feel and/or clean feel, while maintaining conditioning benefits such as slippery feel during the application to wet hair and moisturized feel on dry hair.

Preferably, the composition of the present invention is substantially free of anionic surfactants and anionic polymers, in view of stability of the gel matrix. In the present invention, “substantially free of anionic surfactants and anionic polymers” means that the composition contains 1% or less, preferably 0.5% or less, more preferably totally 0% of total of anionic surfactants and anionic polymers.

### Cationic Surfactant System

The composition of the present invention comprises a cationic surfactant system. The cationic surfactant system can be one cationic surfactant or a mixture of two or more cationic surfactants. The cationic surfactant system is included in the composition at a level by weight of from about 0.1% to about 10%, preferably from about 0.5% to about 8%, more preferably from about 1% to about 5%, still more preferably from about 1.5% to about 4%, in view of balance among ease-to-rinse feel and/or clean feel, rheology and wet conditioning benefits.

A variety of cationic surfactants including mono- and di-alkyl chain cationic surfactants can be used in the compositions of the present invention. Among them, preferred are mono-alkyl chain cationic surfactants in view of providing desired gel matrix and wet conditioning benefits. The mono-alkyl cationic surfactants are those having one long alkyl chain which has from 12 to 22 carbon atoms, preferably from 16 to 22 carbon atoms, more preferably C18-22 alkyl group, in view of providing balanced wet conditioning benefits. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms. Such mono-alkyl cationic surfactants include, for example, mono-alkyl quaternary ammonium salts and mono-alkyl amines. Mono-alkyl quaternary ammonium salts include, for example, those having a non-functionalized long alkyl chain. Mono-alkyl amines include, for example, mono-alkyl amidoamines and salts thereof.

Mono-long alkyl quaternized ammonium salts useful herein are those having the formula (II):

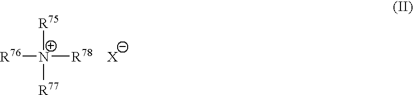

wherein one of R⁷⁵, R⁷⁶R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 22 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Examples of preferred mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt. Among them, highly preferred are behenyl trimethyl ammonium salt and stearyl trimethyl ammonium salt.

Mono-alkyl amines are also suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamindopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearaminde. Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid. The amines herein are preferably partially neutralized with any of the acids at a molar ratio of the amine to the acid of from about 1:0.3 to about 1:2, more preferably from about 1:0.4 to about 1:1.

Although the mono-alkyl chain cationic surfactants are preferred, other cationic surfactants such as di-alkyl chain cationic surfactants may also be used alone, or in combination with the mono-alkyl chain cationic surfactants. Such di-alkyl chain cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

### High Melting Point Fatty Compound

The composition of the present invention comprises a high melting point fatty compound. The high melting point fatty compound is included in the composition at a level of from about 1% to about 15%, preferably from about 3% to about 10%, more preferably from about 5% to about 8% by weight of the composition, in view of providing improved conditioning benefits such as slippery feel during the application to wet hair, and moisturized feel on dry hair.

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about 22 carbon atoms. These fatty alcohols are saturated and can be straight or branched chain alcohols. Preferred fatty alcohols include, for example, cetyl alcohol, stearyl alcohol, behenyl alcohol, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

### Aqueous Carrier

The conditioning composition of the present invention comprises an aqueous carrier. The level and species of the carrier are selected according to the compatibility with other components, and other desired characteristic of the product. Generally, the compositions of the present invention comprise from about 20% to about 99%, preferably from about 30% to about 95%, and more preferably from about 80% to about 95% water.

The carrier useful in the present invention includes water and water solutions of lower alkyl alcohols and polyhydric alcohols. The lower alkyl alcohols useful herein are monohydric alcohols having 1 to 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product.

### Gel Matrix

Preferably, the above cationic surfactants, together with high melting point fatty compounds and an aqueous carrier, form a gel matrix in the composition of the present invention.

The gel matrix is suitable for providing various conditioning benefits such as slippery feel during the application to wet hair and moisturized feel on dry hair. In view of providing the above gel matrix, the cationic surfactant and the high melting point fatty compound are contained at a level such that the weight ratio of the cationic surfactant to the high melting point fatty compound is in the range of, preferably from about 1:1 to about 1:10, more preferably from about 1:1 to about 1:6.

For forming gel matrix, it is preferred to prepare the composition by the following method:

Water is typically heated to at least about 70° C., preferably between about 80° C. and about 90° C. The cationic surfactant and the high melting point fatty compound are combined with the water to form a mixture. The temperature of the mixture is preferably maintained at a temperature higher than both the melting temperature of the cationic surfactant and the melting temperature of the high melting point fatty compound, and the entire mixture is homogenized. After mixing until no solids are observed, the mixture is gradually cooled (e.g., at a rate of from about 1° C./minute to about 5° C./minute) to a temperature below 60° C., preferably less than about 55° C. During this gradual cooling process, a significant viscosity increase is observed at between about 55° C. and about 65° C. This indicates the formation of gel matrix. The high molecular weight water-soluble cationic polymer can be added to the mixture with agitation at about 55° C., or prior to the cooling down. Additional components are then combined with the gel matrix, and cooled to room temperature.

Preferably, the present invention comprises, by weight of the composition, from about 60% to about 99%, preferably from about 70% to about 95%, and more preferably from about 80% to about 95% of a gel matrix, to which optional ingredients such as silicones can be added. The composition containing the above amount of gel matrix is typically characterized by a viscosity of from about 1,000 cps to about 50,000 cps, preferably from about 5,000 cps to about 40,000 cps, and more preferably from about 10,000 cps to about 30,000 cps, as measured at 25° C., by means of a Brookfield Viscometer at shear rate of 1.0 rpm. Although the composition of the present invention may contain a thickening polymer, the composition of the present invention can have the above viscosity without the presence of any thickening polymer.

### Silicone Agent

The composition of the present invention comprises a silicone agent comprising a silicone backbone, hydrophobic portion and hydrophilic portion. It has been surprisingly found that this silicone agent provides ease-to-rinse feel and/or clean feel, increased rheology, and wet softness benefit, while maintaining conditioning benefits such as slippery feel during the application to wet hair and moisturized feel on dry hair. Without intending to be limited by theory, it is believed that: the silicone agent is interacted into the gel matrix because of the hydrophobic portion especially preferred hydrophobic portion which is similar to fatty compounds resulting in higher rheology; and ease-to-rinse feel and/or clean feel benefit is obtained by the fast breakdown of gel matrix when rinsing, due to the hydrophilic/polar portion of the silicone agent. This silicone active agent also provides softness on the wet hair by depositing on the hair.

The silicone agent is included in the composition at a level by weight of from about 0.01% to about 10%, preferably from about 0.05% to about 5%, more preferably from about 0.1% to about 3%, in view of balance among ease-to-rinse feel and/or clean feel, rheology and wet conditioning benefits.

The hydrophobic portions are, for example, those having straight or branched alkyl or alkoxy group having from 3 to 22 carbon atoms, preferably from 8 to 18 carbon atoms, more preferably from 10 to 16 carbon atoms. Among them, preferred are alkoxy group having preferably from 8 to 18 carbon atoms, more preferably from 10 to 16 carbon atoms.

The hydrophilic portions are, for example, amino group, alkyl amino group, amido group, alkyl amido group, polyol group, and mixtures thereof.

Among them, preferred are alkyl amino group such as alkyl amino group in which the alkyl has from 1 to 10 carbon atoms, more preferably from 3 to 5 carbon atoms, polyol group such as propylene glycol, and mixtures thereof.

The hydrophobic portion and hydrophilic portion can attach to the silicone backbone respectively. Alternatively, the hydrophobic and hydrophilic portions can be in one substitution group, the substitution group being attached to the silicone backbone. In this invention, it is preferred that one or both ends, more preferably both ends, of the silicone backbone are terminated by the hydrophobic groups, while the hydrophilic group is attached to silicone backbone as side chain (pendent group).

Highly preferred silicone agents are those having the following formula:

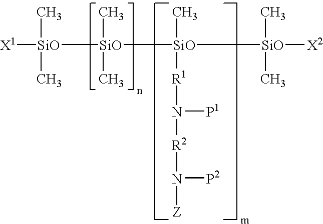

wherein n is an integer of 1 or above, preferably 50 or above; and m is an integer of 1 or above, preferably 5 or above; the sum of n and m is a number from 10 to 2000, preferably from 100 to 1000; X¹ and X² are, respectively, C3-C22 alkoxy, preferably C8 to C18 alkoxy, more preferably C10-C16 alkoxy; P¹ and P² are, respectively, hydrogen, propylene glycol, polypropylene glycol, or polyethylene oxide, preferably propylene glycol; R¹ and R² are, respectively, C1-C10 alkyl groups, preferably straight, non-functionalized C2-C8 alkyl group, more preferably straight, non-functionalized C3-C5 alkyl group; Z is P² or that having the following formula: —C(O)—R³—P³, preferably Z is P². In the formula —C(O)—R³—P³, R³ is C1-C22 alkyl groups, preferably straight, non-functionalized C3-C18 alkyl group, more preferably straight, non-functionalized C5-C16 alkyl group, and P³ is hydrogen, hydroxyoxide, propylene glycol, polypropylene glycol, or polyethylene oxide, preferably hydroxide. Such highly preferred silicone agents include, for example; those having an INCI name Bis (C13-15 Alkoxy) PG Amodimethicone available, for example, with a tradename DC 8500 from Dow Corning; those having an INCI name Bis(C13-15 Alkoxy) Hydroxybutamidoamodimethicone available, for example, with a tradename DC 8813 from Dow Corning; and those having an INCI name Bis(C13-15 Alkoxy)

Hydroxybutamidoamodimethicone available, for example, with a tradename DC 8803 from Dow Corning.

The silicone agents useful herein have a viscosity of preferably, from about 100 cst to about 100,000 cst, more preferably from about 500 cst to about 50,000 cst, still more preferably from about 1,000 cst to about 30,000 cst, in view of providing ease-to-rinse feel and/or clean feel, increased rheology and wet softness.

### Silicone Conditioning Compound

Preferably, the compositions of the present invention contain a silicone conditioning compound. The silicone conditioning compound useful herein are not polymers described above under the tile “SILICONE AGENT”.

It is believed that the silicone conditioning compound can provide smoothness and softness on dry hair. The silicone conditioning compounds herein can be used at levels by weight of the composition of preferably from about 0.1% to about 20%, more preferably from about 0.15% to about 10%, still more preferably from about 0.2% to about 8%.

The silicone conditioning compounds useful herein, as a single compound, as a blend or mixture of at least two silicone conditioning compounds, or as a blend or mixture of at least one silicone conditioning compound and at least one solvent, have a viscosity of preferably from about 1,000 to about 2,000,000 mPa·s at 25° C.

The viscosity can be measured by means of a glass capillary viscometer as set forth in Dow Corning Corporate Test Method CTM0004, Jul. 20, 1970. Suitable silicone fluids include polyalkyl siloxanes, polyaryl siloxanes, polyalkylaryl siloxanes, polyether siloxane copolymers, amino substituted silicones, quaternized silicones, and mixtures thereof. Other nonvolatile silicone conditioning compounds having conditioning properties can also be used.

Preferably, the silicone conditioning compounds have an average particle size of from about 1 microns to about 50 microns, in the composition.

The silicone conditioning compounds useful herein include polyalkyl or polyaryl siloxanes with the following structure:

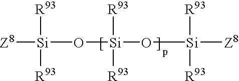

wherein R⁹³ is alkyl or aryl, and p is an integer from about 7 to about 8,000. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the hair, is compatible with the other components of the composition, is chemically stable under normal use and storage conditions, and is capable of being deposited on and conditions the hair. Suitable Z⁸ groups include hydroxy, methyl, methoxy, ethoxy, propoxy, and aryloxy. The two R⁹³ groups on the silicon atom may represent the same group or different groups. Preferably, the two R⁹³ groups represent the same group. Suitable R⁹³ groups include methyl, ethyl, propyl, phenyl, methylphenyl and phenylmethyl. The preferred silicone conditioning compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone conditioning compounds are available, for example, from the General Electric Company in their Viscasil® and TSF 451 series, and from Dow Corning in their Dow Corning SH200 series.

The above polyalkylsiloxanes are available, for example, as a mixture with silicone conditioning compounds having a lower viscosity. Such mixtures have a viscosity of preferably from about 1,000 mPa·s to about 100,000 mPa·s, more preferably from about 5,000 mPa·s to about 50,000 mPa·s. Such mixtures preferably comprise: (i) a first silicone having a viscosity of from about 100,000 mPa·s to about 30,000,000 mPa·s at 25° C., preferably from about 100,000 mPa·s to about 20,000,000 mPa·s; and (ii) a second silicone having a viscosity of from about 5 mPa·s to about 10,000 mPa·s at 25° C., preferably from about 5 mPa·s to about 5,000 mPa·s. Such mixtures useful herein include, for example, a blend of dimethicone having a viscosity of 18,000,000 mPa·s and dimethicone having a viscosity of 200 mPa·s available from GE Toshiba, and a blend of dimethicone having a viscosity of 18,000,000 mPa·s and cyclopentasiloxane available from GE Toshiba.

The silicone conditioning compounds useful herein also include a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone conditioning compounds. This overlap is not intended as a limitation on any of these materials. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. The silicone gums are available, for example, as a mixture with silicone conditioning compounds having a lower viscosity. Such mixtures useful herein include, for example, Gum/Cyclomethicone blend available from Shin-Etsu.

The silicone conditioning compounds that can be used include, for example, a polypropylene oxide modified polydimethylsiloxane although ethylene oxide or mixtures of ethylene oxide and propylene oxide can also be used. The ethylene oxide and polypropylene oxide level should be sufficiently low so as not to interfere with the dispersibility characteristics of the silicone. These materials are also known as dimethicone copolyols.

Silicone conditioning compounds useful herein also include amino substituted materials. Preferred aminosilicones include, for example, those which conform to the general formula (III):

(R₁)ₐG₃₋ₐ—Si—(—OSiG₂)ₙ—(—OSiG_{b}(R₁)_{2-b})ₘ—O—SiG₃₋ₐ(R₁)ₐ

wherein G is hydrogen, phenyl, hydroxy, or C₁-C₈ alkyl, preferably methyl; a is 0 or an integer having a value from 1 to 3, preferably 1; b is 0, 1 or 2, preferably 1; n is a number from 0 to 1,999; m is an integer from 0 to 1,999; the sum of n and m is a number from 1 to 2,000; a and m are not both 0; R₁ is a monovalent radical conforming to the general formula CqH_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A; wherein R₂ is hydrogen, phenyl, benzyl, or a saturated hydrocarbon radical, preferably an alkyl radical from about C₁ to about C₂₀; A is a halide ion.

One highly preferred amino silicones are those corresponding to formula (III) wherein m=0, a=1, q=3, G=methyl, n is preferably from about 1500 to about 1700, more preferably 1600; and L is —N(CH₃)₂ or —NH₂, more preferably —NH₂. Another highly preferred amino silicones are those corresponding to formula (III) wherein m=0, a=1, q=3, G=methyl, n is preferably from about 400 to about 600, more preferably about 500; and L is —N(CH₃)₂ or —NH₂, more preferably —NH₂. Such highly preferred amino silicones can be called as terminal aminosilicones, as one or both ends of the silicone chain are terminated by nitrogen containing group.

The above aminosilicones, when incorporated into the composition, can be mixed with solvent having a lower viscosity. Such solvents include, for example, polar or non-polar, volatile or non-volatile oils. Such oils include, for example, silicone oils, hydrocarbons, and esters. Among such a variety of solvents, preferred are those selected from the group consisting of non-polar, volatile hydrocarbons, volatile cyclic silicones, non-volatile linear silicones, and mixtures thereof. The non-volatile linear silicones useful herein are those having a viscosity of from about 1 to about 20,000 centistokes, preferably from about 20 to about 10,000 centistokes at 25° C. Among the preferred solvents, highly preferred are non-polar, volatile hydrocarbons, especially non-polar, volatile isoparaffins, in view of reducing the viscosity of the aminosilicones and providing improved hair conditioning benefits such as reduced friction on dry hair. Such mixtures have a viscosity of preferably from about 1,000 mPa·s to about 100,000 mPa·s, more preferably from about 5,000 mPa·s to about 50,000 mPa·s.

Other suitable alkylamino substituted silicone conditioning compounds include those represented by the following structure:

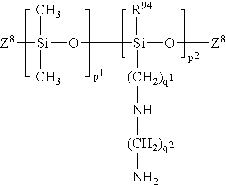

wherein R⁹⁴ is H, CH₃ or OH; p¹ and p² are integers of 1 or above, and wherein sum of p¹ and p² is from 650 to 1,500; q¹ and q² are integers of from 1 to 10. Z⁸ represents groups which block the ends of the silicone chains. Suitable Z⁸ groups include hydroxy, methyl, methoxy, ethoxy, propoxy, and aryloxy. Highly preferred are those known as “amodimethicone”. Commercially available amodimethicones useful herein include, for example, BY16-872 available from Dow Corning.

The silicone conditioning compounds may further be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and mixtures thereof.

### Thickening Polymer System

The compositions of the present invention may contain a thickening polymer. The thickening polymers useful herein are those which can provide the composition with an appropriate viscosity of from about 1,000 cps to about 50,000 cps, preferably from about 5,000 cps to about 40,000 cps, and more preferably from about 10,000 cps to about 30,000 cps, as measured at 25° C., by means of a Brookfield Viscometer at shear rate of 1.0 rpm.

The thickening polymer herein can be used at levels by weight of the composition of from about 0.05% to about 5%, preferably from about 0.1% to about 3%.

A variety of thickening polymers can be used in the compositions of the present invention. Thickening polymers useful herein include, for example, cellulose and its derivatives such as cellulose ethers, hydrophobically modified cellulose ethers, and quaternized celluloses; nonionic guar gums; cationic guar gums; crosslinked polymers such as nonionic crosslinked polymers and cationic crosslinked polymers; and acrylate polymers such as sodium polyacrylate, polyethylacrylate, and polyacrylamide. The thickening polymers useful herein may include the polymers disclosed below under the title “CATIONIC POLYMER”. Among a variety of thickening polymers, preferred are nonionic guar gums.

The nonionic guar polymer useful herein has a molecular weight of preferably from about 100,000 AMU (Atomic Mass Unit) to about 4,000,000 AMU, more preferably from about 500,000 AMU to about 3,500,000 AMU, still more preferably from about 1,000,000 AMU to about 3,000,000 AMU, even more preferably from about 1,600,000 AMU to about 2,800,000 AMU. Commercially available nonionic guar polymers useful herein include, for example, that having a molecular weight of about 2,000,000 AMU and having a tradename Jaguar HP-105 available from Rhodia, and N-hance HP series such as 40 and 40 S available from Aqualon.

### Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as panthenol available from Roche, panthenyl ethyl ether available from Roche, hydrolysed collagen with tradename Peptein 2000 available from Hormel, vitamin E with tradename Emix-d available from Eisai, hydrolysed keratin, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; perfumes; and sequestering agents, such as disodium ethylenediamine tetra-acetate; ultraviolet and infrared screening and absorbing agents such as octyl salicylate, octyl methoxycinnamate, benzophenone-3 and nenzophenone-4; and antidandruff agents such as zinc pyrithione and salicylic acid.

### Low Melting Point Oil

Low melting point oils useful herein are those having a melting point of less than 25° C. The low melting point oil useful herein is selected from the group consisting of: hydrocarbon having from 10 to about 40 carbon atoms; unsaturated fatty alcohols having from about 10 to about 30 carbon atoms such as oleyl alcohol; unsaturated fatty acids having from about 10 to about 30 carbon atoms; fatty acid derivatives; fatty alcohol derivatives; ester oils such as pentaerythritol ester oils, trimethylol ester oils, citrate ester oils, and glyceryl ester oils; poly α-olefin oils; and mixtures thereof. Preferred low melting point oils herein are selected from the group consisting of: ester oils such as pentaerythritol ester oils, trimethylol ester oils, citrate ester oils, and glyceryl ester oils; poly α-olefin oils; and mixtures thereof.

Particularly useful pentaerythritol ester oils and trimethylol ester oils herein include pentaerythritol tetraisostearate, pentaerythritol tetraoleate, trimethylolpropane triisostearate, trimethylolpropane trioleate, and mixtures thereof.

Particularly useful poly α-olefin oils herein include polydecenes with tradenames PURESYN 6 having a number average molecular weight of about 500 and PURESYN 100 having a number average molecular weight of about 3000 and PURESYN 300 having a number average molecular weight of about 6000 available from Exxon Mobil Co.

### Polyethylene Glycol

Polyethylene glycol can also be used as an additional component. The polyethylene glycols useful herein that are especially preferred are PEG-2M wherein an average value of ethylene glycol units is about 2,000; PEG-SM wherein an average value of ethylene glycol units is about 5,000; PEG-7M wherein an average value of ethylene glycol units is about 7,000; PEG-9M wherein an average value of ethylene glycol units is about 9,000; and PEG-14M wherein an average value of ethylene glycol units is about 14,000.

### Cationic Conditioning Polymer

Cationic conditioning polymers useful herein are those having an average molecular weight of at least about 5,000, typically from about 10,000, preferably from about 100,000 to about 3 million.

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. Other suitable spacer monomers include vinyl esters, vinyl alcohol (made by hydrolysis of polyvinyl acetate), maleic anhydride, propylene glycol, and ethylene glycol. Other suitable cationic polymers useful herein include, for example, cationic celluloses, cationic starches, and cationic guar gums.

### Product Forms

The conditioning compositions of the present invention can be in the form of rinse-off products or leave-on products, and can be formulated in a wide variety of product forms, including but not limited to creams, gels, emulsions, mousses and sprays.

The conditioning composition of the present invention is especially suitable for rinse-off hair conditioner. Such compositions are preferably used by following steps:

(i) after shampooing hair, applying to the hair an effective amount of the conditioning compositions for conditioning the hair; and
(ii) then rinsing the hair.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

[Compositions]

| Components | Ex. 1 | Ex. 2 | Ex. 3 | Ex. 4 | Ex. 5 | Ex. 6 | Ex. 7 | Ex. 8 | Ex. 9 |
|---|---|---|---|---|---|---|---|---|---|
| Stearamidopropyl | 1 | 1.2 | 0.8 | 1 | 1 | 1 | 2 | — | — |
| Dimethylamine | | | | | | | | | |
| L-Glutamic acid | — | — | — | — | — | — | 0.64 | | |
| Ditallow dimethyl ammonium | 0.75 | 0.95 | 0.95 | 0.75 | 0.75 | 0.75 | — | — | — |
| chloride | | | | | | | | | |
| Behenyl trimethyl ammonium | — | — | — | — | — | — | — | 2.85 | 2.85 |
| chloride | | | | | | | | | |
| Cetyl alcohol | 1.2 | 1.0 | 1.2 | 1.2 | 1.5 | 0.96 | 2.5 | 1.9 | 1.9 |
| Stearyl alcohol | 0.8 | 0.7 | 1.0 | 0.8 | 0.6 | 0.64 | 4.5 | 4.6 | 4.6 |
| Silicone active agent-1 *1 | 0.25 | 0.5 | 0.5 | — | — | 0.6 | 0.7 | 1.0 | 0.5 |
| Silicone active agent-2 *2 | — | — | — | 0.5 | — | — | — | — | — |
| Silicone active agent-3 *3 | — | — | — | — | 0.5 | — | — | — | — |
| Aminosilicone *4 | 0.4 | 0.5 | 0.7 | 0.3 | 0.8 | — | — | 1.5 | 3.5 |
| Dimethicone/Cyclomethicone *5 | — | — | — | — | — | 4.2 | 4.2 | — | — |
| PEG-2M | — | 0.2 | — | — | — | 0.5 | — | — | — |
| Oleyl alcohol | 0.25 | 0.25 | 0.25 | 0.25 | 0.25 | 0.25 | — | — | — |
| Hydroxypropyl guar *6 | 0.3 | — | — | 0.3 | 0.3 | — | — | — | — |
| Hydroxyethyl cellulose | — | 0.25 | 0.25 | — | — | 0.25 | — | — | — |
| Citric Acid | 0.12 | 0.12 | 0.12 | 0.12 | 0.12 | 0.13 | — | — | — |
| Acid EDTA | 0.1 | 0.1 | 0.1 | 0.1 | 0.1 | 0.1 | 0.1 | — | — |
| Disodium EDTA | — | — | — | — | — | — | — | 0.13 | 0.13 |
| Benzyl alcohol | 0.4 | 0.4 | 0.4 | 0.4 | 0.4 | 0.4 | 0.4 | 0.4 | 0.4 |
| Kathon CG *7 | 0.03 | 0.03 | 0.03 | 0.03 | 0.03 | 0.03 | 0.03 | 0.03 | 0.03 |
| Perfume | 0.40 | 0.40 | 0.40 | 0.40 | 0.40 | 0.40 | 0.40 | 0.40 | 0.40 |
| Panthenol | 0.03 | 0.03 | 0.03 | — | 0.03 | 0.03 | 0.03 | 0.03 | 0.03 |
| Panthenyl ethyl ether | 0.03 | 0.03 | 0.03 | — | 0.03 | 0.03 | 0.03 | 0.03 | 0.03 |
| Sodium Hydroxide | — | — | — | — | — | — | — | 0.014 | 0.014 |

| | |
|---|---|
| Deionized Water | q.s. to 100% |

Definitions of Components
*1 Silicone active agent-1: Amodimethicone having an INCI name Bis (C13-15 Alkoxy) PG Amodimethicone available with a tradename DC 8500 from Dow Corning having a viscosity of about 2,500 cSt.
*2 Silicone active agent-2: Bis(C13-15 Alkoxy) Hydroxybutamidoamodimethicone available with tradename DC 8813 from Dow Corning having a viscosity of about 5,000 cSt.
*3 Silicone active agent-3: Bis(C13-15 Alkoxy) Hydroxybutamidoamodimethicone available with tradename DC 8803 from Dow Corning having a viscosity of about 8,000 cSt.
*4 Aminosilicone: Terminal aminosilicone which is available from GE having a viscosity 10,000 mPa · s, and having following formula (III): (R₁)ₐG₃₋ₐ—Si—(—OSiG₂)ₙ—(—OSiG_{b}(R₁)_{2-b})ₘ—O—SiG₃₋ₐ(R₁)ₐ (III) wherein G is methyl; a is an integer of 1; b is 0, 1 or 2, preferably 1; n is a number from 400 to about 600; m is an integer of 0; R₁ is a monovalent radical conforming to the general formula CqH_{2q}L, wherein q is an integer of 3 and L is —NH₂
*5 Dimethicone/Cyclomethicone: a blend dimethicone having a viscosity of 18,000,000 mPa · s and cyclopentasiloxane available from GE Toshiba
*6 Hydroxypropyl guar: Jaguar HP-105 available from Rhodia
*7 Kathon CG: Available from Rohm&Haas

### Method of Preparation

The conditioning compositions of “Ex. 1” through “Ex. 9” as shown above can be prepared by any conventional method well known in the art. They are suitably made as follows:

Cationic surfactants and high melting point fatty compounds are added to water with agitation, and heated to about 80° C. The mixture is cooled down to about 55° C. Silicone active agent is added to the mixture. If included, silicone conditioning compounds, perfumes, preservatives are added to the mixture with agitation. Then the mixture is cooled down to room temperature.

Examples 1 through 9 are hair conditioning compositions of the present invention which are particularly useful for rinse-off use. The embodiments disclosed and represented by the previous “Ex. 1” through “Ex. 9” have many advantages. For example, they can provide ease-to-rinse feel and/or clean feel, increased rheology, and wet softness while maintaining improved conditioning benefits of gel matrix such as slippery feel during the application to wet hair. They can also provide such ease-to-rinse feel and/or clean feel while remaining a sufficient amount of deposition of conditioning agents on the hair to provide dry conditioning benefits such as moisturized feel on dry hair.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the same term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A conditioning composition comprising by weight:
(a) from about 0.1% to about 10% of a cationic surfactant;
(b) from about 1% to about 15% of a high melting point fatty compound;
(c) from about 0.01% to about 10% of a silicone agent comprising a silicone backbone, a hydrophilic portion, and a hydrophobic portion; and
(d) an aqueous carrier.

2. The conditioning composition of claim 1 wherein the hydrophobic portion is selected from the group consisting of straight or branched alkyl or alkoxy group having from 3 to 22 carbon atoms, and wherein the hydrophilic portion is selected from the group consisting of amino group, alkyl amino group, amido group, alkyl amido group, polyol group, and mixtures thereof.

3. The conditioning composition of claim 2 wherein the hydrophobic portion is selected from the group consisting of straight or branched alkyl or alkoxy group having from 8 to 18 carbon atoms, and wherein the hydrophilic portion is selected from the group consisting of alkyl amino group in which the alkyl has from 1 to 10 carbon atoms, polyol group, and mixtures thereof.

4. The conditioning composition of claim 3 wherein the silicone agent is the hydrophobic portion is straight or branched alkoxy group having from 10 to 16 carbon atoms, and wherein the hydrophilic portion is selected from the group consisting of alkyl amino group in which the alkyl has from 3 to 5 carbon atoms, propylene glycol, and mixtures thereof.

5. The conditioning composition of claim 1 wherein one or both ends of the silicone backbone are terminated by the hydrophobic portions, and wherein the hydrophilic group is attached to silicone backbone as a side chain.

6. The conditioning composition of claim 1 wherein the silicone agent has a viscosity of from about 100 cst to about 100,000 cst.

7. The conditioning composition of claim 1 wherein the cationic surfactant is selected from mono-alkyl cationic surfactants.

8. The conditioning composition of claim 1 further comprising from about 0.1% to about 20% of a silicone conditioning compound.

9. The conditioning composition of claim 1 wherein the composition is substantially free of anionic surfactants and anionic polymers.

10. The conditioning composition of claim 1 which is a rinse-off hair conditioning composition.
